(19) United States
(12) Patent Application Publication
(10) Pub. No.: US 2025/0104571 A1
(43) Pub. Date: Mar. 27, 2025

(54) METHOD AND SYSTEM FOR ASSESSING DEVELOPMENTAL AGE OF CHILDREN

(71) Applicants: MacKay Memorial Hospital, Taipei City (TW); National Taipei University of Technology, Taipei City (TW)

(72) Inventors: Hui-Ju CHEN, Taipei City (TW); Tun-Wen PAI, Taipei City (TW); Hsin-Ju Mary KO, Taipei City (TW)

(21) Appl. No.: 18/372,700

(22) Filed: Sep. 26, 2023

(51) Int. Cl. G09B 7/04; A61B 5/16

(52) U.S. Cl. CPC G09B 7/04; A61B 5/16; A61B 2503/06

(57) ABSTRACT

Disclosed herein is an improved system and methods implemented by the system for producing an assessment on the developmental age of a child through evaluation of six developmental domains. The method comprises steps of: (a) selecting a first set of questions to produce a first question list that corresponds to a first age range; (b) calculating a first score of the first question list; (c) producing a second question list based on the first calculated score of step (b) by selecting a second set of questions from the selected question pool of step (a); (d) calculating a second score of the second question list; (e) determining a developmental age of the child in the corresponded developmental domain based on the second calculated score of step (d); and (f) repeating steps (a) to (e), thereby producing the assessment. Also disclosed herein is a method for treating the child having a developmental delay by providing an early intervention to the child.

### BACKGROUND OF THE INVENTION

### 1. Field of the Invention

The present disclosure relates to a method and system for assessing a developmental age of a child. More specifically, the present disclosure pertains to a novel method for determining a developmental delay (DD) of the child by evaluating multiple developmental domains, and treating the child with DD by an early intervention.

### 2. Description of Related Art

The incidence of developmental delays in children is increasing year by year. In addition to congenital factors, the impact of postnatal environmental factors leading to developmental delays is also a significant concern. The effectiveness of treating developmental delays in children depends on whether caregivers identify the issue and seek assistance early, enabling relevant therapeutic interventions to address the problem. However, changes in family composition and caregiving patterns in society nowadays have also introduced unfavorable environmental factors that could accelerate the developmental delays.

Child development assessment tools currently used in clinics are relatively limited and simplified. They are effective only for individuals who already exhibit significant signs of developmental delays, but they fall short in conducting assessments and monitoring during the initial stages of development. Moreover, the lack of a comprehensive and integrated long-term tracking system hinders the efficient utilization of medical and social resources across various diverse locations.

In view of the foregoing, there exists in the related art a need for a novel system for comprehensive assessment of child development, and a method for treating children in need through an early intervention.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, the purpose of the present disclosure is to provide a novel system and method for determining a developmental age of a child through assessing six developmental domains with the aid of a feedback loop. As a result, the system of the present disclosure can offer precise information about children's neurological development in a timely manner, and an appropriate treatment for a child in need can be timely intervened.

In one aspect, the present disclosure is directed to a system for producing an assessment on the development age of a child with the aid of evaluation in six developmental domains. The system comprises a user interface configured to receive an answer inputted externally; a storage unit configured to provide six question pools respectively corresponding to the six developmental domains, wherein each question pool comprises a plurality of questions independently corresponding to an age range from 0 to 72 months old; and a processor communication linked to the user interface and the storage unit.

According to embodiments of the present disclosure, and configured to execute the instructions programmed therein by implementing the actions of: (a) selecting a first set of questions corresponding the same age range from one of the question pools stored in the storage unit to produce a first question list that corresponds to a first age range; (b) calculating a first score of the first question list based on an externally inputted answer for the first question list of step (a); (c) producing a second question list based on the first calculated score of step (b) by selecting a second set of questions from the selected question pool of step (a), wherein the second set of questions differ from the first set of questions, and the second question list corresponds to a second age range ascending to or descending from the first age range; (d) calculating a second score of the second question list based on the externally inputted answer for the second question list of step (c); (e) determining a developmental age of the child in the corresponded developmental domain based on the second calculated score of step (d); and (f) repeating steps (a) to (e) until the developmental age for each developmental domain have been collected, thereby producing the assessment on the developmental age of the child. In preferred working examples, each age range differs from the next age range by two months.

In some working examples, in the step (a) of the action implemented by the processor, the first age range covers the chronological age of the child.

According to one embodiment of the present disclosure, in the step (c) of the action implemented by the processor, when the first calculated score of step (b) is smaller than a standard score, the second age range corresponding to the second question list descends from the first age range.

Accordingly, after step (d) of the action, when the first calculated score of step (b) is smaller than the standard score, the action further comprises (d-i) repeating producing at least one new question list by selecting a new set of questions different from the first and second sets of questions until the calculated score thereof is larger than the standard score for the first time, wherein the new question list corresponds a new age range descending from the previous one. Further, in step (e) of the action implemented by the processor, the developmental age of the child in the corresponded developmental domain is determined based on the new age range corresponding to the new question list. Specifically, if the developmental age of the child is younger than the chronological age of the child, then the child is determined to have a developmental delay (DD) in the corresponded developmental domain.

According to another embodiment of the present disclosure, in the step (c) of the action implemented by the processor, when the first calculated score of step (b) is larger than the standard score, the second age range corresponding to the second question list ascends to the first age range.

Accordingly, in step (d) of the action implemented by the processor, when the first calculated score of step (b) is larger than the standard score, then the developmental age of the child is determined based on the second calculated score of step (d), and the developmental age is equivalent to the second age range corresponding to the second question list of step (c).

According to some working embodiments, in step (e), if the second calculated score of step (d) is smaller than the standard score, the developmental age of the child is determined to be matched with the chronological age of the child. According to other working embodiments, in step (e), if the second calculated score of step (d) is larger than the standard score, the developmental age of the child is determined to be older than the chronological age of the child.

According to some embodiments of the present disclosure, the processor of the preset system is configured to further implement the action of performing an automatic detection for reliability and validity assessment of steps (a) to (e). In one working example, the automatic detection is conducted by performing a deep learning algorithm.

In some embodiments of the present disclosure, the first and the second sets of questions respectively have a number no more than four.

In another aspect, the present disclosure pertains to a method for determining and treating a development delay (DD) of a child through evaluation of six developmental domains. The method comprises: (a) providing six question pools respectively corresponding to the six developmental domains, wherein each question pool comprises a plurality of questions independently corresponding to an age range from 0 to 72 months old; (b) selecting a first set of questions corresponding to the same age range from one of the question pools of step (a) to produce a first question list that corresponds to a first age range; (c) calculating a first score of the first question list based on an answer received for the first question list of step (b); (d) producing a second question list based on the first calculated score of step (c) by selecting a second set of questions from the selected question pool of step (b), wherein the second set of questions differ from the first set of questions, and the second question list corresponds to a second age range ascending to or descending from the first age range; (e) calculating a second score of the second question list based on an answer received for the second question list of step (d); (f) determining a developmental age of the child in the corresponded developmental domain based on the second calculated score of step (e); (g) repeating steps (b) to (f) until the developmental ages for all six developmental domains have been collected, thereby determining the degree of the DD of the child; and (h) providing an early intervention to treat the DD of the child based on the degree of the DD determined in step (g).

In some working examples, each age range differs from the next age range by two months.

In other working examples, the first age range covers the chronological age of the child.

According to one embodiment of the present disclosure, the first set and second set of questions respectively have a number no more than four.

According to one embodiment of the present disclosure, in step (d) of the present method, when the first calculated score of step (c) is smaller than a standard score, the second age range corresponding to the second question list descends from the first age range.

Accordingly, after step (e), the present method further comprises (e-i) when the first calculated score of step (c) is smaller than the standard score, repeating producing at least one new question list by selecting a new set of questions different from the first and second sets of questions, until the calculated score thereof is larger than the standard score for the first time, wherein the new question list corresponds a new age range descending from the previous one.

Accordingly, in step (f) of the present method, the developmental age of the child in the corresponded developmental domain is determined based on the new age range corresponding to the new question list. In preferred working example, if the developmental age of the child is younger than the chronological age of the child, then the child is determined to have the DD in the corresponded developmental domain.

According to another embodiment of the present disclosure, when the first calculated score of step (c) is larger than the standard score, hen the second age range corresponding to the second question list ascends to the first age range.

Accordingly, in some preferred embodiments, when the first calculated score of step (c) is larger than the standard score, the step (f) of the present method further comprises (f-i) determining a developmental age of the child based on the second calculated score of step (e) wherein, the developmental age is equivalent to the second age range corresponding to the second question list of step (d).

According to one working example of the present disclosure, in step (f-i), if the second calculated score of step (e) is smaller than the standard score, the developmental age of the child is determined to be matched with the chronological age of the child. Alternatively, still in step (f-i), if the second calculated score of step (e) is larger than the standard score, the developmental age of the child is determined to be older than the chronological age of the child.

According to some embodiments of the present disclosure, examples of the early intervention of step (h) comprises a speech-language therapy, an occupational therapy (OT), a physical therapy (PT), a behavioral therapy, an early childhood special education (ECSE), a sensory integration therapy, a psychotherapy, an alternative and augmentative communication (AAC), and a combination thereof.

In some alternative or optional embodiments of the present disclosure, the method further comprises performing an automatic detection for reliability and validity assessment of steps (b) to (f). In some working examples, the automatic detection is conducted by performing a deep learning algorithm.

By virtue of the above configuration, the system and method for making an assessment for children's developmental age and treating a child having a development delay can offer timely and accurate information about children's development, thereby improving the efficiency and accuracy in early intervention of a developmental delayed child.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a schematic diagram illustrating a system 10 according to one embodiment of the present disclosure; and

FIG. 2 is a flow chart of a method 20 implemented on the system 10 according to one embodiment of the present disclosure.

In accordance with common practice, the various described features/elements are not drawn to scale but instead are drawn to best illustrate specific features/elements relevant to the present invention. Also, like reference numerals and designations in the various drawings are used to indicate like elements/parts.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. Definition

For convenience, certain terms employed in the specification, examples and appended claims are collected here. Unless defined otherwise, all technical and scientific terms used herein have the same meaning as commonly understood by one of the ordinary skilled in the art to which this invention belongs.

The singular forms “a”, “and”, and “the” are used herein to include plural referents unless the context clearly dictates otherwise.

The term “child” or “children” as used herein refers to a human being who is in the early stages of development, typically under six years old. The term of a “child” as used in the present disclosure intends to encompass an infant from birth to 12 months of age, a toddler from 1 to 3 years old, a preschooler who is between 3 and 5 years old, and a school-age of 6 years old.

The term “age range” as used herein refers to a specific time interval defined within a certain period. According to embodiments of the present disclosure, the age ranges are equally spaced intervals within the range of 0 to 72 months (i.e., under six years old). For example, each interval can be one month, two months, or three months, and there is no overlap between each age range and the subsequent one. According to one working example, the age range is two months.

The term “developmental domain” as used herein refers to different aspect used to measure various facets of children's physiological and psychological growth and development. Exemplary developmental domains mainly encompass physical, cognitive, language, and social-emotional domains. According to embodiments of the present disclosure, the six developmental domains used in the present assessment are specifically gross motor skills, fine motor skills, language comprehension, language expression, cognition, and social-emotional development.

The term “developmental age” as used herein refers to the developmental age determined by the present system for children who have undergone the developmental assessment set forth in the present disclosure. The determination is based on the answers provided by the assessed child in response to the questions that reflect the developmental stage exhibited in each of six developmental domains. Depending on the assessment, the developmental age may or may not match with the child's actual physiological age (i.e., chronological age).

The term “development delay(s) (DD)” as used herein encompasses not only clinically discernible signs or symptoms of developmental disorders, but also includes conditions where, as assessed through the present system and/or method of the present disclosure, a child has not reached the developmental state expected for their actual chronological age. Specifically, after assessment by the present method and/or system, if the developmental age of a specific developmental domain lags behind the child's chronological age, he/she would be identified as having a developmental delay (DD).

As used herein, the term “treat,” “treating” and “treatment” are interchangeable, and encompasses partially or completely intervening, preventing, ameliorating, mitigating and/or managing a sign, symptom, a disorder or a condition associated with development delays in a child.

### 2. Description of the Invention

Clinically, the developmental status of children is evaluated through six developmental domains, including gross motor skills, fine motor skills, language comprehension, language expression, cognition, and social-emotional development. To enhance the accuracy of assessment results, the present invention aims to provide a comprehensive assessment system with the ability of self-assess reliability and validity feedback, thereby facilitating timely monitoring and early intervention. Also herein is a method for determining and treating a development delay (DD) of a child with the aid of the aforementioned system and method.

### 2.1 System and Method for Accessing a Developmental Age of a Child

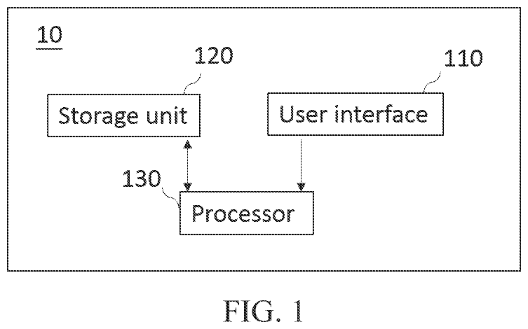

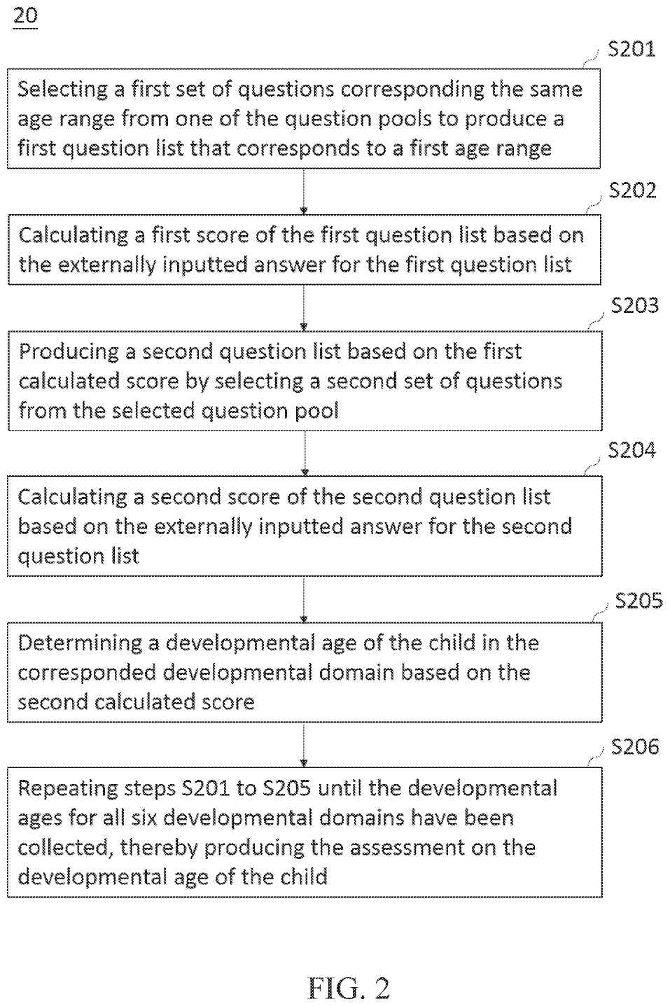

One aspect of the present disclosure is to provide a system and a method for accessing a developmental age of a child. References are made to FIGS. 1 and 2.

FIG. 1 is a schematic drawing depicting a system 10 for implementing a method 20 of the present disclosure, while FIG. 2 is a flow chart of the method 20 executed by the present system 10. The system 10 comprises a user interface 110, a storage unit 120, and a processor 130, wherein the user interface 110 and the storage unit 120 are respectively coupled to the processor 130. According to embodiments of the present disclosure, the user interface 110 is configured to receive an answer inputted externally by a user. In certain embodiments of the present disclosure, the user interface 110 exists as a computer window or an online webpage. The storage unit 120 is configured to store six question pools respectively corresponding to six developmental domains, and each question pool comprises a plurality of questions. The processor 130 is configured to execute the instructions programmed therein to produce an assessment on the developmental age of a child with the aid of the plurality of questions from each question pool stored in the storage unit 120.

In some embodiments, the storage unit 120 is configured to storage databases composed of multiple questionnaires. Examples of the storage unit 120 suitable for use in the present disclosure include, but are not limited to, a hard disk drive, a solid-state drive, an external drive, an online storage, a blockchain storage and the like. The processor 130 is programmed with instructions to execute the present method for assessing a developmental age for a child, which will be described in detail in subsequent paragraphs. Preferably, the user interface 110, the storage unit 120, and the processor 130 are integrated into one hardware. Alternatively, they may exist as individual devices located at various places and commutatively connected to each other via network, such as by a local area network (LAN), a wide area network (WAN), the Internet, or a wireless network. According to certain embodiments, the user interface 110 may be in the form of an online webpage, allowing external users (e.g., clinicians, parents, educators, and therapists) to interact with the present system 10 and/or method 20.

Referring to FIG. 2, which depicts a flow chart of the method 20 executed by the system 10 for producing an assessment on the developmental age of a child with the aid of evaluation in six developmental domains. The method 20 includes the following steps (see the reference numbers S201 to S206 indicated in FIG. 2),
S201: selecting a first set of questions corresponding to the same age range from one of the question pools stored in a storage unit to produce a first question list that corresponds to a first age range;
S202: calculating a first score of the first question list based on an externally inputted answer for the first question list of step S201;
S203: producing a second question list based on the first calculated score of step S202 by selecting a second set of questions from the selected question pool of step S201, wherein the second set of questions differ from the first set of questions, and the second question list corresponds to a second age range ascending to or descending from the first age range;
S204: calculating a second score of the second question list based on an externally inputted answer for the second question list of step S203;
S205: determining a developmental age of the child in the corresponded developmental domain based on the second calculated score of step S204; and
S206: repeating steps S201 to S205 until the developmental ages for all six developmental domains have been collected, thereby producing the assessment on the developmental age of the child.

According to embodiments of the present disclosure, the six developmental domains are gross motor skills, fine motor skills, language comprehension, language expression, cognition, and social-emotional development, respectively. The plurality of questions individually corresponding to each developmental domain are created in advance based on expertise in the related art and clinical knowledge, and stored as individual question pools in the storage unit 120 of the present system 10.

According to embodiments of the present disclosure, each questions stored in the storage unit 120 independently corresponds to an age range from 0 month old to 72 months old, such as, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 month(s) old. The age range of the present disclosure is an interval of several months within the ages between 0 month to 72 months, such as the interval of one, two, three, four, five, six, eight, ten, or twelve month(s). In some embodiments of the present disclosure, the age range is two months within the ages between 0 month to 72 months; preferably, each age range differs from the next age range by two months. In one working example, the age range is from 0 to 2 months old, from 2 to 4 months old, from 4 to 6 months old, from 6 to 8 months old, from 8 to 10 months old, from 10 to 12 months old, from 12 to 14 months old, from 14 to 16 months old, from 16 to 18 months old, from 18 to 20 months old, from 20 to 22 months old, from 22 to 24 months old, from 24 to 26 months old, from 26 to 28 months old, from 28 to 30 months old, from 30 to 32 months old, from 32 to 34 months old, from 34 to 36 months old, from 36 to 38 months old, from 38 to 40 months old, from 40 to 42 months old, from 42 to 44 months old, from 44 to 46 months old, from 46 to 48 months old, from 48 to 50 months old, from 50 to 52 months old, from 52 to 54 months old, from 54 to 56 months old, from 56 to 58 months old, from 58 to 60 months old, from 60 to 62 months old, from 62 to 64 months old, from 64 to 66 months old, from 66 to 68 months old, from 68 to 70 months old, and from 70 to 72 months old.

Thus, in step S201, a first set of questions is selected from one of the question pools that corresponds to one of the developmental domains, and all the selected questions therein corresponds to the same age range between 0 to 72 months old set forth above, thereby producing a first question list that corresponds to a first age range. The first age range can be determined in accordance with practical needs; in one working example, the first age range encompasses the chronological ages of the tested child.

According to some embodiments of the present disclosure, the first question list is presented through the user interface 110 of the system 10, and external responses to the questions (i.e., the answers to the questions inputted by users) are collected via the user interface 110 as well. Then, the method 20 executed by the processor 130 will proceed to a calculation step, wherein a first score of the first question list is calculated based on the collected answers (step S202).

According to some preferred embodiments of the present disclosure, in step S202, a standard score for every sets of questions may be determined in advance based on the general knowledge and criteria well known in the art. Thus, the first score calculated in step S202, whether with or without weighting, is compared with a standard score, thereby producing a second question list based on the comparison result (S203). In one embodiment, the second question list is produced by selecting a second set of questions from the same question pool in step S201, notably the second set of questions differ from the first set of questions, and the second question list corresponds to a second age range different from the first age range (step S203). According to embodiments of the present disclosure, the second age range is ascending to or descending from the first age range. In one working example, when the first calculated score in step S202 is smaller than the standard score, then the second question list of which the second age range descends from the first age range is produced and presented to the user through the same user interface 110 (S203). Alternatively, when the first calculated score in step S202 is equal to and/or larger than the standard score, then the second question list of which the second age range ascends to the first age range is produced (S203) and presented to the user though the user interface 110.

Similar to step S202, in S204, the second question list is displayed via the user interface 110. Responses entered externally from the user are collected through the same user interface 110 and transmitted to the processor 130, enabling the processor 130 to calculate a second score. After the second score is calculated, a developmental age of the child in the corresponding developmental domain is determined (S205).

In some embodiments of the present disclosure, the first and second set of questions selected to produce the first and second question lists may comprise same or different numbers of questions. Preferably, each set of questions contains no more than four questions; more preferably, no more than three questions. In one working example, the first and second sets of question independently contains four questions.

According to embodiments of the present disclosure, the present method and/or system may determine the developmental age of each child undergoing the evaluation based on the comparison between the calculated score and the standard score via executing one of the two options (I) and (II) described below.

(I) The First Calculated Score Smaller than the Standard Score

In the case when the first calculated score of step S202 is smaller than the standard score, then a second question list having a second age range descending from the first age range is provided. At this point, the second score for the second question list is verified, and a new set of questions differ from those previously answered is selected from the same question pool to produce a new question list. Specifically, if the second calculated score is still smaller than the standard score, then the present method will proceed to select a new set of questions (e.g., a third set of questions) from the same question pool as set forth above in steps S203 to produce another new question list (e.g., a third question list). According to embodiments of the present disclosure, the criteria for selecting the third set of questions is the same as those described in steps S201 or S203, wherein the age range of the third question list descends further from the second question lists. If the third score is still smaller than the standard score, the present method will continue to produce another new question list until the calculated score of any given question list is larger than that of the standard score for the first time. In other words, after repeating the steps of selecting the age range-descending question sets several times, the questions selection process stops once a selected question list with a score greater than the standard score appears. At this point, the age range corresponding to the selected question list is determined as the developmental age of the tested child.

Under this process, if the child's developmental age is younger than his/her chronological age, then the child is identified as having a developmental delay (DD) in the respective developmental domain.

(II) The First Calculated Score Larger than, or Equal to the Standard Score

Alternatively, in the case when the first calculated score of step S202 is larger than the standard score, than the present method will proceed to produce a second question list, of which the second age range ascends to the first age range (i.e., the second age range is older than the first age range) (step S203). After receiving externally inputted answers for the second question list and calculating the second score, the developmental age of the child is determined based on the comparison between the second score and the standard score. In one working example, the second score of step S204 is smaller than the standard score, and the developmental age of the child is determined to be matched with the chronological age of the child. In an alternative working example, the second score calculated in step S204 is larger than the standard score, and the developmental age of the child is determined to be older than the chronological age of the child. Under this approach, the child's developmental age in one developmental domain is obtained.

The strategies outlined in option (I) or (II) are independently carried out by sequentially addressing the questions selected from the six developmental domains for the tested child. By repeating steps S201 to S205, the developmental age for each developmental domain is gathered to generate an assessment on the child's developmental age (step S206).

In addition or optionally, the present method may further implement an automatic assessment on the reliability and validity of steps S201 to S205, such that the credibility of the selected questions are verified. The reliability and validity assessment can be automatically conducted by tools and algorithms well known in the art in accordance with practical needs. In some working examples, the automatic assessment is conducted by performing a deep learning algorithm. Examples of the deep learning algorithm suitable for use in the present system and method include, but are not limited to, neural network, decision tree, random forest, logistic regression, support vector machine, principal component analysis, k-means clustering, and a combination thereof. In one working example, the automatic assessment is conducted by support vector machine.

### 2.2 Methods for Determining and Treating Development Delay

The present disclosure also aims at providing treatment to a child determined as having a development delay (DD). To this purpose, the afore-described method and system may be utilized to assist clinicians in making a precise determination on a child's developmental age so that the child identified to be having DD may be timely treated (e.g., via administering a treatment). The present disclosure thus encompasses a method for determining and treating a development delay (DD) of a child under 72 months old (or six years old).

According to some embodiments of the present disclosure, the method comprises,
(a) providing six question pools respectively corresponding to the six developmental domains, wherein each question pool comprises a plurality of questions independently corresponding to an age range from 0 to 72 months old;
(b) selecting a first set of questions corresponding to the same age range from one of the question pools of step (a) to produce a first question list that corresponds to a first age range;
(c) calculating a first score of the first question list based on an answer received for the first question list of step (b);
(d) producing a second question list based on the first calculated score of step (c) by selecting a second set of questions from the selected question pool of step (b), wherein the second set of questions differ from the first set of questions, and the second question list corresponds to a second age range ascending to or descending from the first age range;
(e) calculating a second score of the second question list based on an answer received for the second question list of step (d);
(f) determining a developmental age of the child in the corresponded developmental domain based on the second calculated score of step (e);
(g) repeating steps (b) to (f) until the developmental age for each developmental domain is collected, thereby determining the degree of the DD of the child; and
(h) providing an early intervention to treat the DD of the child based on the result of step (g).

The present method begins by providing six question pools respectively corresponding to six developmental domains, and each question pool comprises a plurality of questions, which may be created or designed for verifying the development of a child. According to one working example of the present disclosure, the six question pools are stored in the storage unit 120 of the present system 10. Then, a first set of questions corresponding to the same age range is selected from one of the six question pools to produce a first question list that corresponds to a first age range (step (a)). Note that in one preferred working example, the first age range covers the child's chronological age. Base on the first score calculated from answers inputted by the tested child for the first question list, a second question list that corresponds to another appropriate age range is further produced. Subsequently, based on the second score calculated from answers to the second question list, the child's developmental age is eventually determined via procedures described above in section 2.1 of this paper.

According to embodiments of the present disclosure, question lists from the six question pools (note that each pool corresponds to one of the six developmental domains) are supposed to be tested by repeating steps (b) to (f) set forth above, thereby comprehensively determining the degree of the DD of the tested child. Specifically, the proportion of DD within the six developmental domains can be determined as one-sixth, two-sixths, three-sixths, four-sixths, five-sixths or all of the domains is DD.

Once a child is assessed to have a DD in any of the developmental domain, appropriate intervention should be initiated, and the degree of the DD (e.g., 1/6, 2/6, 3/6 and etc.) of the child determined and confirmed by the present method is used as a reference for the determination of which early intervention should be applied to the child. Examples of the early intervention suitable for use in the present method (i.e., for providing to a child having DD) include, but are not limited to, a speech-language therapy, an occupational therapy (OT), a physical therapy (PT), a behavioral therapy, an early childhood special education (ECSE), a sensory integration therapy, a psychotherapy, an alternative and augmentative communication (AAC), and a combination thereof. Any clinical artisans may choose a suitable treatment for use in the present method based on factors such as the particular condition being treated, the severity of the condition, the individual patient parameters (including age, physical condition, and gender), the duration of the treatment, the nature of concurrent therapy (if any), the specific environments and like factors within the knowledge and expertise of the health practitioner. According to some embodiments of the present disclosure, in step (h), if the child exhibits DD in any of the developmental domain, an early intervention through any combination of the aforementioned treatment is required. For example, if a two year-old child is determined to have a DD of 2/6 in domains of language comprehension and language expression, then a combination of speech-language therapy and physical therapy is applied to the child.

By the virtue of the above features, the present method can provide precise assessment and determination of developmental delay for a child, thereby improving accuracy and efficiency of early treatment and intervention for the development delayed child.

### EXAMPLES

### Example 1 Evaluation of the Present Method for Producing Developmental Assessment

The purpose of this experiment is to validate the performance and accuracy of the present system and method for conducting developmental assessments. Accordingly, statistical analysis is employed to analyze the assessing outcomes across six developmental domains and the whole system. Results are summarized in Tables 1 and 2, respectively.

TABLE 1
Verified efficiency results of the present system in developmental
assessment for six developmental domains

| | | | | Language | |
|---|---|---|---|---|---|
| Gross motor skills | | Fine motor skills | | comprehension | |
| Accuracy | 0.717 | Accuracy | 0.63 | Accuracy | 0.739 |
| Sensitivity | 0.644 | Sensitivity | 0.558 | Sensitivity | 0.524 |
| Specificity | 0.787 | Specificity | 0.694 | Specificity | 0.92 |
| Precision | 0.717 | Precision | 0.615 | Precision | 0.846 |
| F1 score | 0.678542 | F1 score | 0.585115 | F1 score | 0.647159 |
| | | | | Social-emotional | |
| Language expression | | Cognition | | development | |
| Accuracy | 0.728 | Accuracy | 0.76 | Accuracy | 0.782 |
| Sensitivity | 0.667 | Sensitivity | 0.485 | Sensitivity | 0.79 |
| Specificity | 0.816 | Specificity | 0.915 | Specificity | 0.767 |
| Precision | 0.837 | Precision | 0.762 | Precision | 0.875 |
| F1 score | 0.742392 | F1 score | 0.592735 | F1 score | 0.83033 |

TABLE 2
Verified efficiency results of the present whole system
The present system (%)

| | |
|---|---|
| Accuracy | 92 |
| Sensitivity | 93.3 |
| Specificity | 66.7 |
| Precision | 98.8 |
| F1 score | 95.9 |

The results depicted in Tables 1 and 2 confirm that the present system exhibits high accuracy in assessing developmental stages of children, either within individual domain or a whole system.

By using the present method and system, the developmental ages of children can be automatically determined and identified, thereby improving the efficiency and accuracy in diagnosis and early intervention for developmental delay.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for determining and treating a development delay (DD) of a child through evaluation of six developmental domains, comprising:
(a) providing six question pools respectively corresponding to the six developmental domains, wherein each question pool comprises a plurality of questions independently corresponding to an age range from 0 to 72 months old;
(b) selecting a first set of questions corresponding to the same age range from one of the question pools of step (a) to produce a first question list that corresponds to a first age range;
(c) calculating a first score of the first question list based on an answer received for the first question list of step (b);
(d) producing a second question list based on the first calculated score of step (c) by selecting a second set of questions from the selected question pool of step (b), wherein the second set of questions differ from the first set of questions, and the second question list corresponds to a second age range ascending to or descending from the first age range;
(e) calculating a second score of the second question list based on an answer received for the second question list of step (d);
(f) determining a developmental age of the child in the corresponded developmental domain based on the second calculated score of step (e);
(g) repeating steps (b) to (f) until the developmental ages for all six developmental domains have been collected, thereby determining the degree of the DD of the child; and
(h) providing an early intervention to treat the DD of the child based on the degree of the DD determined in step (g).

2. The method of claim 1, wherein in step (d),
when the first calculated score of step (c) is smaller than a standard score, the second age range corresponding to the second question list descends from the first age range; or
when the first calculated score of step (c) is larger than the standard score, the second age range corresponding to the second question list ascends to the first age range.

3. The method of claim 2, further comprising, after step (e),
(e-i) when the first calculated score of step (c) is smaller than the standard score, repeating producing at least one new question list by selecting a new set of questions different from the first and second sets of questions, until the calculated score of the new question list is larger than the standard score for the first time, wherein the new question list corresponds a new age range descending from the previous one.

4. The method of claim 3, wherein in step (f), the developmental age of the child in the corresponded developmental domain is determined based on the new age range corresponding to the new question list.

5. The method of claim 4, wherein in step (f), if the developmental age of the child is younger than a chronological age of the child, then the child is determined to have the DD in the corresponded developmental domain.

6. The method of claim 2, wherein in step (f) in the case that the first calculated score of step (c) is larger than the standard score, further comprising
(f-i) determining a developmental age of the child based on the second calculated score of step (e), wherein, the developmental age is equivalent to the second age range corresponding to the second question list of step (d).

7. The method of claim 6, wherein in step (f-i), if the second calculated score of step (e) is smaller than the standard score, the developmental age of the child is determined to be matched with a chronological age of the child.

8. The method of claim 6, wherein in step (f-i), if the second calculated score of step (e) is larger than the standard score, the developmental age of the child is determined to be older than a chronological age of the child.

9. The method of claim 1, wherein the early intervention of step (h) is selected from the group consisting of a speech-language therapy, an occupational therapy (OT), a physical therapy (PT), a behavioral therapy, an early childhood special education (ECSE), a sensory integration therapy, a psychotherapy, an alternative and augmentative communication (AAC), and a combination thereof.

10. The method of claim 1, further comprising performing an automatic detection for reliability and validity assessment of steps (b) to (f).

11. The method of claim 10, wherein the automatic detection is conducted by performing a deep learning algorithm.

12. The method of claim 1, wherein the first set and second set of questions respectively have a number no more than four.

13. The method of claim 1, wherein each age range differs from the next age range by two months.

14. The method of claim 1, wherein in step (b), the first age range covers a chronological age of the child.
